(19) United States
(12) Patent Application Publication
(10) Pub. No.: US 2026/0345594 A1
(43) Pub. Date: Nov. 5, 2026

(54) Methods and Compositions for Controlling Jet Lag, Chronotype, or Circadian Rhythm

(71) Applicants: University of Pittsburgh - Of the Commonwealth System of Higher Education, Pittsburgh, PA (US); The Research Foundation for The State University of New York, Albany, NY (US)

(72) Inventors: Stephen Yu-Wah Chan, Pittsburgh, PA (US); Colleen A. McClung, Gibsonia, PA (US); Ivet Bahar, Setauket, NY (US); Hongying Cheng, Pittsburgh, PA (US); Jiyoung Lee, Wexford, PA (US)

(21) Appl. No.: 19/107,764

(22) PCT Filed: Sep. 1, 2023
(86) PCT No.: PCT/US23/31883

§ 371 Date: Feb. 28, 2025

Related U.S. Application Data
(60) Provisional application No. 63/374,459, filed on Sep. 2, 2022.

(51) Int. Cl. A61K 31/498; A61K 31/404; A61K 31/4365

(52) U.S. Cl. CPC A61K 31/498; A61K 31/404; A61K 31/4365

(57) ABSTRACT

Methods, compositions, and devices are provided for use in controlling jet lag, chronotype, and/or circadian rhythm in a patient. Patients can be treated with a vasopressin 1A receptor antagonist or a melanopsin agonist to control the patient's jet lag, chronotype, and/or circadian rhythm.

### CROSS REFERENCE TO RELATED APPLICATIONS

This application claims priority to U.S. Provisional Patent Application No. 63/374,459 filed Sep. 2, 2022, the disclosure of which is hereby incorporated herein by reference in its entirety.

Jet lag remains a significant problem for individuals who frequently travel or work variable night and day shifts. Jet lag is caused by a misalignment between an individual's internal, biologically driven circadian clock and the environmental light/dark cycle. There are limited options to counteract jet lag, and current treatments are not the strongest synchronizers of rhythms, they take time to work, and they don't work equally well for everyone. Much of the work in circadian rhythm for the purpose of controlling jet lag has been to target the molecular clock that is inherent to all cells in the body. However, the ability to alter the perceived shifts in light/dark cycles in the body has not been fully realized. Along with controlling or treating jet lag, methods of controlling a patient's chronotype or circadian rhythm are desired.

### SUMMARY

A method of reducing or controlling jet lag, modifying chronotype, and/or modulating a circadian clock of a patient, e.g., a human patient, is provided, comprising: administering a vasopressin 1A receptor antagonist to the patient in an amount effective to reduce or control jet lag, modify chronotype, and/or modulate a circadian clock of the patient; and/or administering a melanopsin agonist to the patient in an amount effective to reduce or control jet lag, modify chronotype, and/or modulate a circadian clock of the patient.

A composition is provided, comprising, a vasopressin 1A receptor antagonist and/or a melanopsin agonist, in amounts effective to reduce or control jet lag, modify chronotype, and/or modulate a circadian clock of the patient, and a pharmaceutically-acceptable excipient.

A melanopsin agonist compound is provided, having the structure (iv):

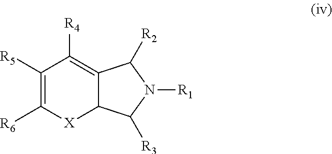

wherein,
R₁ is an aromatic five-membered heterocycle group, optionally linked by a methylene group, comprising one or both of N and O, such as furanyl and oxazolyl, including 1,2-oxazolyl and 1,3-oxazolyl, as well as methylfuranyl and methyloxazolyl, including methyl-1,2-oxazolyl and methyl-1,3-oxazolyl,
   R₂ is oxygen (carbonyl) or phenyl or halo-substituted phenyl, such as o, m, or p-fluoro phenyl,
   R₃ is oxygen or indolyl,
   R₄ is H or oxygen,
   R₅ and R₆ are both H, or together form a phenyl or naphthyl group, and
   X is C or O,
      or a pharmaceutically-acceptable salt thereof, and
      wherein the melanopsin agonist does not comprise MolPort-002-983-412 or MolPort-000-525-975.

A vasopressin 1A receptor antagonist is provided, having the structure (i):

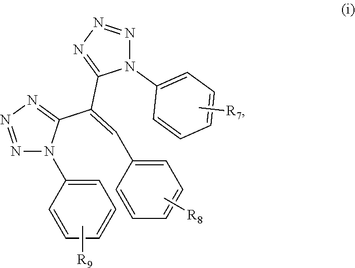

wherein R₇, R₈, and R₉ are, independently, H, hydroxyl, methyl, or nitro, and in one example, at least one of R₇, R₈, and R₉ may be OH, methyl, or nitro, or a pharmaceutically-acceptable salt thereof, or
a compound having the structure (ii):

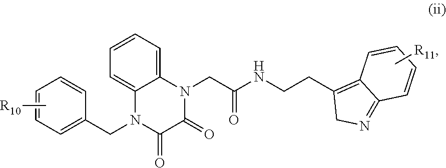

wherein R₁₀ and R₁₁ are, independently: H, hydroxyl, or methyl, or a pharmaceutically-acceptable salt thereof, and
wherein the vasopressin 1A receptor antagonist does not comprise MolPort-002-260-938 and MolPort-007-880-661.

The following numbered clauses outline various non-limiting aspects and embodiments of the invention.

Clause 1. A method of reducing or controlling jet lag, modifying chronotype, administering and/or modulating a circadian clock of a patient, e.g., a human patient, comprising:
a vasopressin 1A receptor antagonist to the patient in an amount effective to reduce or control jet lag, modify chronotype, and/or modulate a circadian clock of the patient; and/or
administering a melanopsin agonist to the patient in an amount effective to reduce or control jet lag, modify chronotype, and/or or modulate a circadian clock of the patient.

Clause 2. The method of clause 1, wherein the patient is a human patient.

Clause 3. The method of clause 1 or 2, comprising administering a vasopressin 1A receptor antagonist to the patient in an amount effective to reduce or control jet lag, modify chronotype, and/or modulate a circadian clock of the patient.

Clause 4. The method of any one of clauses 1-3, wherein the vasopressin 1A receptor antagonist is selected from the group consisting of: ZINC257318833, MolPort-010-769-806, MolPort-010-769-803, MolPort-004-227-511, MolPort-009-703-593, MolPort-009-703-587, MolPort-003-361-612, MolPort-000-810-610, MolPort-019-855-958, ZINC100959508, MolPort-007-684-659, MolPort-007-880-661, MolPort-002-641-911, MolPort-010-799-948, MolPort-003-073-533, MolPort-002-260-938, MolPort-006-539-702, ZINC24843601, ZINC20611382, ZINC16845443, ZINC10233808, ZINC20611384, ZINC20610887, ZINC62728312, ZINC12231390, or a pharmaceutically acceptable salt thereof.

Clause 5. The method of any one of clauses 1-3, wherein the vasopressin 1A receptor antagonist is selected from MolPort-007-880-661, MolPort-003-073-533, MolPort-002-260-938, or a pharmaceutically-acceptable salt thereof.

Clause 6. The method of any one of clauses 1-3, wherein the vasopressin 1A receptor antagonist comprises a compound, having the structure (i):

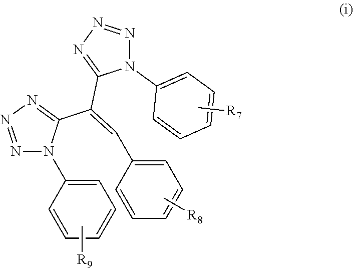

wherein R₇, R₈, and R₉ are, independently, H, hydroxyl, methyl, or nitro, and optionally at least one of R₇, R₈, and R₉ are OH, methyl, or nitro, or a pharmaceutically-acceptable salt thereof.

Clause 7. The method of clause 6, wherein R₇ and R₉ are H, and R₈, is nitro (e.g. MolPort-002-260-938), or a pharmaceutically-acceptable salt thereof.

Clause 8. The method of any one of clauses 1-3, wherein the vasopressin 1A receptor antagonist comprises a compound, having the structure (ii):

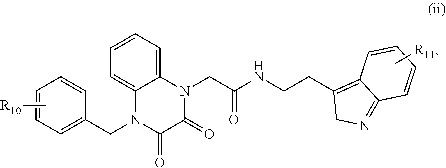

wherein R₁₀ and R₁₁ are, independently: H, hydroxyl, or methyl, or a pharmaceutically-acceptable salt thereof.

Clause 9. The method of any one of clauses 1-3, wherein the vasopressin 1A receptor antagonist comprises a compound, having the structure (ii, e.g., MolPort-007-880-661):

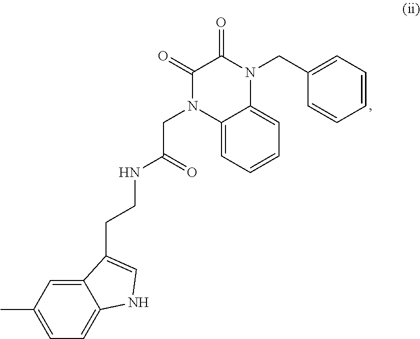 [SMILES: Cc1ccc2[nH]cc(CCNC(=O)Cn3c(=O)c(=O)n(Cc4ccccc4)c4ccccc43)c2c1]

or a pharmaceutically-acceptable salt thereof.

Clause 10. The method of clause 1 or 2, comprising administering a melanopsin agonist to the patient in an amount effective to reduce or control jet lag, modify chronotype, and/or or modulate a circadian clock of the patient.

Clause 11. The method of clause 10, wherein the melanopsin agonist comprises a compound selected from the group consisting of: MolPort-046-192-342, MolPort-046-751-265, MolPort-027-820-910, MolPort-039-341-893, MolPort-000-786-730, MolPort-004-901-380, MolPort-002-739-585, MolPort-005-950-241, MolPort-047-468-801, MolPort-005-950-837, MolPort-047-768-812, MolPort 002-120-903, ZINC91651216, ZINC270133816, ZINC585287286, ZINC000334163086, ZINC000027795402, ZINC000000974767, ZINC000016952484, ZINC000002762284, ZINC000009487830, ZINC000034034721, and ZINC000015972050, or a pharmaceutically acceptable salt thereof.

Clause 12. The method of clause 10, wherein the melanopsin agonist comprises MolPort-002-983-412, or a pharmaceutically-acceptable salt thereof.

Clause 13. The method of clause 10, wherein the melanopsin agonist comprises MolPort-000-525-975, or a pharmaceutically-acceptable salt thereof.

Clause 14 The method of clause 10, wherein the melanopsin agonist comprises a compound having the structure (iv):

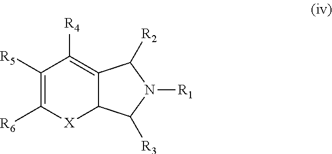

wherein,
R₁ is an aromatic five-membered heterocycle group, optionally linked by a methylene group, comprising one or both of N and O, such as furanyl and oxazolyl, including 1,2-oxazolyl and 1,3-oxazolyl, as well as methylfuranyl and methyloxazolyl, including methyl-1,2-oxazolyl and methyl-1,3-oxazolyl,
R₂ is oxygen (carbonyl) or phenyl or halo-substituted phenyl, such as o, m, or p-fluoro phenyl,
R₃ is oxygen or indolyl,
R₄ is H or oxygen,
R₅ and R₆ are both H, or together form a phenyl or naphthyl group, and
X is C or O,
   or a pharmaceutically-acceptable salt thereof.

Clause 15. The method of clause 14, wherein the melanopsin agonist comprises a compound having the structure:

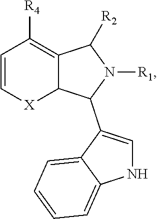

or a pharmaceutically-acceptable salt thereof.

Clause 16. The method of clause 14, wherein the melanopsin agonist comprises a compound having the structure:

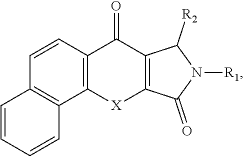

or a pharmaceutically-acceptable salt thereof.

Clause 17. The method of any one of clauses 1-16, comprising: administering both a vasopressin 1A receptor antagonist and a melanopsin agonist to the patient, in amounts effective to reduce or control jet lag, modify chronotype, and/or or modulate a circadian clock of the patient.

Clause 18. A composition, comprising, a vasopressin 1A receptor antagonist and/or a melanopsin agonist, in amounts effective to reduce or control jet lag, modify chronotype, and/or or modulate a circadian clock of the patient, and a pharmaceutically-acceptable excipient.

Clause 19. The composition of clause 18, comprising a vasopressin 1A receptor antagonist.

Clause 20. The composition of clause 19, wherein the vasopressin 1A receptor agonist is selected from the group consisting of: ZINC257318833, MolPort-010-769-806, MolPort-010-769-803, MolPort-004-227-511, MolPort-009-703-593, MolPort-009-703-587, MolPort-003-361-612, MolPort-000-810-610, MolPort-019-855-958, ZINC100959508, MolPort-007-684-659, MolPort-007-880-661, MolPort-002-641-911, MolPort-010-799-948, MolPort-003-073-533, MolPort-002-260-938, MolPort-006-539-702, ZINC24843601, ZINC20611382, ZINC16845443, ZINC10233808, ZINC20611384, ZINC20610887, ZINC62728312, ZINC12231390, or a pharmaceutically acceptable salt thereof.

Clause 21. The composition of clause 19, wherein the vasopressin 1A receptor antagonist is selected from MolPort-007-880-661, MolPort-003-073-533, MolPort-002-260-938, or a pharmaceutically-acceptable salt thereof.

Clause 22. The composition of clause 19, wherein the vasopressin 1A receptor antagonist comprises a compound, having the structure (i):

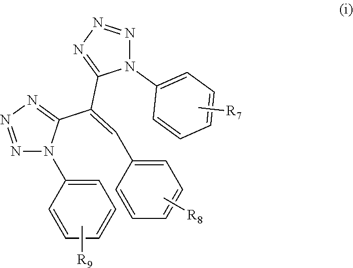

wherein R₇, R₈, and R₉ are, independently, H, hydroxyl, methyl, or nitro, and optionally at least one of R₇, R₈, and R₉ are OH, methyl, or nitro, or a pharmaceutically-acceptable salt thereof.

Clause 23. The composition of clause 22, wherein the vasopressin 1A receptor antagonist does not comprise MolPort-002-260-938 and MolPort-007-880-661.

Clause 24. The composition of clause 22, wherein R₇ and R₉ are H, and R₈, is nitro (e.g. MolPort-002-260-938), or a pharmaceutically-acceptable salt thereof.

Clause 25. The composition of clause 19, comprising a compound having the structure (ii):

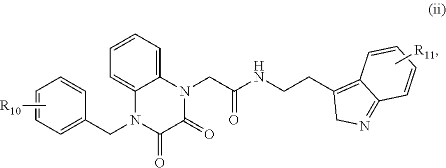

wherein R₁₀ and R₁₁ are, independently: H, hydroxyl, or methyl, or a pharmaceutically-acceptable salt thereof.

Clause 26. The composition of clause 19, wherein the vasopressin 1A receptor antagonist comprises a compound, having the structure (iii, MolPort-007-880-661):

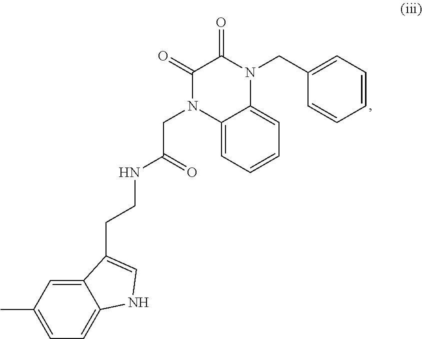 [SMILES: Cc1ccc2[nH]cc(CCNC(=O)Cn3c(=O)c(=O)n(Cc4ccccc4)c4ccccc43)c2c1]

or a pharmaceutically-acceptable salt thereof.

Clause 27. The composition of clause 18, comprising a melanopsin agonist.

Clause 28. The composition of clause 27, wherein the melanopsin agonist comprises a compound selected from the group consisting of: MolPort-046-192-342, MolPort-046-751-265, MolPort-027-820-910, MolPort-039-341-893, MolPort-000-786-730, MolPort-004-901-380, MolPort-002-739-585, MolPort-005-950-241, MolPort-047-468-801, MolPort-005-950-837, MolPort-047-768-812, MolPort-002-120-903, ZINC91651216, ZINC270133816, ZINC585287286, ZINC000334163086, ZINC000027795402, ZINC000000974767, ZINC000016952484, ZINC000002762284, ZINC000009487830, ZINC000034034721, and ZINC000015972050, or a pharmaceutically acceptable salt thereof.

Clause 29. The composition of clause 27, wherein the melanopsin agonist comprises MolPort-002-983-412, or a pharmaceutically-acceptable salt thereof.

Clause 30. The composition of clause 27, wherein the melanopsin agonist comprises MolPort-000-525-975, or a pharmaceutically-acceptable salt thereof.

Clause 31 The composition of clause 27, wherein the melanopsin agonist comprises a compound having the structure (iv):

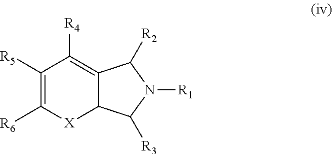

wherein,
R₁ is an aromatic five-membered heterocycle group, optionally linked by a methylene group, comprising one or both of N and O, such as furanyl and oxazolyl, including 1,2-oxazolyl and 1,3-oxazolyl, as well as methylfuranyl and methyloxazolyl, including methyl-1,2-oxazolyl and methyl-1,3-oxazolyl,
R₂ is oxygen (carbonyl), phenyl, or halo-substituted phenyl, such as o, m, or p-fluoro phenyl,
R₃ is oxygen or indolyl,
R₄ is H or oxygen,
R₅ and R₆ are both H, or together form a phenyl or naphthyl group, and
X is C or O,
   or a pharmaceutically-acceptable salt thereof.

Clause 32. The composition of clause 31, wherein the melanopsin agonist comprises a compound having the structure:

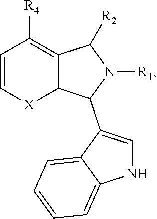

or a pharmaceutically-acceptable salt thereof.

Clause 33. The composition of clause 31, wherein the melanopsin agonist comprises a compound having the structure:

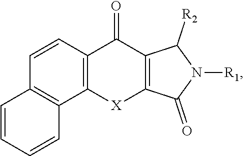

or a pharmaceutically-acceptable salt thereof.

Clause 34. The composition of clause 31, wherein the melanopsin agonist does not comprise MolPort-002-983-412 or MolPort-000-525-975.

Clause 35. The composition of any one of clauses 27-34, in the form of an ocular dosage form.

Clause 36 A melanopsin agonist compound having the structure (iv):

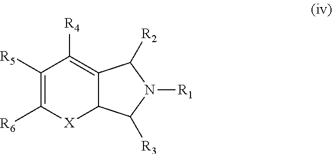

wherein,
R₁ is an aromatic five-membered heterocycle group, optionally linked by a methylene group, comprising one or both of N and O, such as furanyl and oxazolyl, including 1,2-oxazolyl and 1,3-oxazolyl, as well as methylfuranyl and methyloxazolyl, including methyl-1,2-oxazolyl and methyl-1,3-oxazolyl,
R₂ is oxygen (carbonyl), phenyl, or halo-substituted phenyl, such as o, m, or p-fluoro phenyl,
R₃ is oxygen or indolyl,
R₄ is H or oxygen,
R₅ and R₆ are both H, or together form a phenyl or naphthyl group, and
X is C or O,
   or a pharmaceutically-acceptable salt thereof, and
   wherein the melanopsin agonist does not comprise MolPort-002-983-412 or MolPort-000-525-975.

Clause 37. The compound of clause 36, wherein the melanopsin agonist comprises a compound having the structure:

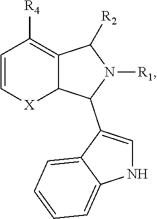

or a pharmaceutically-acceptable salt thereof.

Clause 38. The compound of clause 36, wherein the melanopsin agonist comprises a compound having the structure:

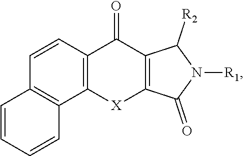

or a pharmaceutically-acceptable salt thereof.

Clause 39. A vasopressin 1A receptor antagonist, having the structure (i):

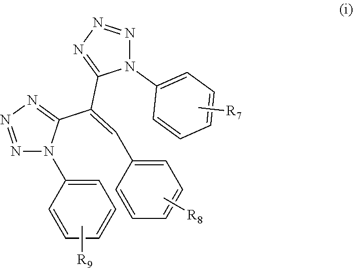

wherein R₇, R₈, and R₉ are, independently, H, hydroxyl, methyl, or nitro, and in one example, at least one of R₇, R₈, and R₉ may be OH, methyl, or nitro, or a pharmaceutically-acceptable salt thereof, or a compound having the structure (ii):

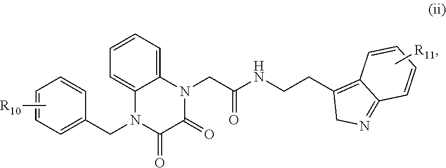

wherein R₁₀ and R₁₁ are, independently: H, hydroxyl, methyl, or a pharmaceutically-acceptable salt thereof, and
wherein the vasopressin 1A receptor antagonist does not comprise MolPort-002-260-938 and MolPort-007-880-661.

Clause 40. A device comprising a dropper bottle and an ocular composition according to a composition of any one of clauses 25-33 contained within the dropper bottle.

### BRIEF DESCRIPTION OF THE DRAWINGS

FIG. 1 is a schematic cross-sectional diagram of an exemplary dropper bottle for ocular delivery of a drug composition as described herein.

FIGS. 2A and 2B. Depict variations of the structure of structure i of Example 1. “H” indicates that the depicted aromatic five-member group comprises one or more hetero-atoms in its ring, such as N and/or O. Groups R₁-R₆ and X are as described in Example 2.

FIGS. 3A and 3B. Pipeline for identifying compounds to inhibit the function of human vasopressin 1a receptor (V1AR). The pipeline includes three major computation components: (A) druggability simulations, (B-C) pharmacophore modeling, and (D) virtual screening. (E) Molecular structures of effective V1AR inhibitors validated by in-vitro experiments.

FIG. 4 shows schematically MD-resolved binding pose of compound 661. The high affinity interacting residues from V1AR are labeled in the black bold face; and weaker interacting residues are colored in gray.

FIG. 5. Computational protocol for identifying compound of melanopsin. The protocol includes three major components: druggability simulations, pharmacophore modeling, and virtual screening.

FIG. 6. Refinement of the lead compound, 975 (MolPort-000-525-975) and 412 (MolPort-002-983-412) by full atomic simulations in the presence of explicit membrane. The interactions between compound atoms and melanopsin residues are shown in the upper 2-dimensional diagrams. Stronger interactions are shown in dark grey solid lines, intermediate interactions are in grey solid lines, and weaker interactions are in dashed lines. At the bottom, three-dimensional binding poses of the compounds are shown bound to the target receptor, melanopsin.

FIGS. 7A-7F provide structures for various compounds described herein.

FIGS. 8A-8F: Identification of the compounds that activate the melanopsin receptor but do not interact with rhodopsin at 5 μM 9-cis-retinal and 16 hrs illumination in a Presto Tango screen. FIG. 8A. Luciferase activity from the Melanopsin Tango is plotted against Log Molport-002-983-412 (Molar). FIG. 8B. Ratio of Luciferase activity over the Positive control plotted against Log Molport-002-983-412 (Molar) showing an EC50 of 7.5 nM. FIG. 8C. Luciferase activity from the Rhodopsin Tango is plotted against Log Molport-002-983-412 (Molar). FIG. 8D. Luciferase activity from the Melanopsin Tango is plotted against Log Molport-000-525-975 (Molar). FIG. 8E Ratio of Luciferase activity over the Positive control plotted against Log Molport-000-525-975 (Molar) showing an EC50 of 0.87 nM. FIG. 8F. Luciferase activity from the Rhodopsin Tango is plotted against Log Molport-000-525-975 (Molar).

FIG. 9: Identification of the compounds that act as allosteric inhibitors of Arginine Vasopressin Receptor AVPR1a at 5 nM Arginine Vasopressin and 16 hrs illumination in a Presto Tango screen. Ratio of Luciferase activity over the Positive control plotted against Log 1) Molport-002-260-938 (Molar) showing an EC50 of 712 nM, 2) Molport-003-073-533 showing an EC50 of 1.0 μM and 3) Molport-007-880-661 showing an EC50 of 360 nM.

### DETAILED DESCRIPTION

The following description is merely exemplary in nature and is in no way intended to limit the invention, its application, or uses. While the description is designed to permit one of ordinary skill in the art to make and use the invention, and specific examples are provided to that end, they should in no way be considered limiting. It will be apparent to one of ordinary skill in the art that various modifications to the following will fall within the scope of the appended claims. The present invention should not be considered limited to the presently disclosed aspects, whether provided in the examples or elsewhere herein.

The use of numerical values in the various ranges specified in this application, unless expressly indicated otherwise, are stated as approximations as though the minimum and maximum values within the stated ranges are both preceded by the word “about”. In this manner, slight variations above and below the stated ranges can be used to achieve substantially the same results as values within the ranges. Also, unless indicated otherwise, the disclosure of these ranges is intended as a continuous range including every value between the minimum and maximum values. For definitions provided herein, those definitions refer to word forms, cognates and grammatical variants of those words or phrases. As used herein “a” and “an” refer to one or more. Patent publications cited below are hereby incorporated herein by reference in their entirety to the extent of their technical disclosure and consistency with the present specification.

As used herein, the terms “comprising,” “comprise” or “comprised,” and variations thereof, are open ended and do not exclude the presence of other elements not identified. In contrast, the term “consisting of” and variations thereof is intended to be closed and excludes additional elements in anything but trace amounts.

As used herein, the term “patient” or “subject” refers to members of the animal kingdom including but not limited to human beings and “mammal” refers to all mammals, including, but not limited to human beings.

As used herein, the “treatment” or “treating” of a patient for a condition, such as controlling jet lag, modifying chronotype, and/or modulating a circadian clock of a patient, e.g., a human patient, means administration to a patient by any suitable dosage regimen, procedure and/or administration route of a composition, device, or structure with the object of achieving a desirable clinical/medical end-point, including but not limited to reducing jet lag and associated symptoms, as well as symptoms of other chronotype or circadian rhythm disorders. An amount of any reagent or therapeutic agent, administered by any suitable route, effective to treat a patient is an amount capable of controlling jet lag, modifying chronotype, and/or or modulating a circadian clock of a patient, e.g., a human patient. The therapeutically-effective amount of each therapeutic may range from 1 μg per dose to 10 g per dose, including any amount there between, such as, without limitation, 1 ng, 1 μg, 1 mg, 10 mg, 100 mg, or 1 g per dose. The therapeutic agent may be administered by any effective route, and, for example, as a single dose or bolus, at regular or irregular intervals, in amounts and intervals as dictated by any clinical parameter of a patient, or continuously.

Active ingredients, such as the compounds described below, exemplified by the described 412, 975, 661, 533, and 938 compounds, may be compounded or otherwise manufactured into a suitable composition for use, such as a pharmaceutical dosage form or drug product in which the compound is an active ingredient.

Compositions may comprise a pharmaceutically acceptable carrier, or excipient. An excipient is an inactive substance used as a carrier for the active ingredients of a medication. Although “inactive,” excipients may facilitate and aid in increasing the delivery or bioavailability of an active ingredient in a drug product. Non-limiting examples of useful excipients include: antiadherents, binders, rheology modifiers, coatings, disintegrants, emulsifiers, oils, buffers, salts, acids, bases, fillers, diluents, solvents, flavors, colorants, glidants, lubricants, preservatives, antioxidants, sorbents, vitamins, sweeteners, etc., as are available in the pharmaceutical/compounding arts.

Useful dosage forms include, for example and without limitation: parenteral, intravenous, intramuscular, intraocular, or intraperitoneal solutions, oral tablets or liquids, topical drops, ointments, or creams, and transdermal devices (e.g., patches).

The compound may be a sterile solution comprising the active ingredient (drug or compound), and a solvent, such as water, saline, lactated Ringer's solution, or phosphate-buffered saline (PBS). Additional excipients, such as polyethylene glycol, emulsifiers, salts and buffers may be included in the solution.

Suitable dosage forms may include single-dose, or multiple-dose vials or other containers, such as medical syringes or droppers, e.g., eye droppers, containing a composition comprising an active ingredient useful for controlling jet lag, modifying chronotype, and/or modulating a circadian clock of a patient, e.g., a human patient, as described herein.

Pharmaceutical formulations adapted for administration include aqueous and non-aqueous sterile solutions which may contain, in addition to the active pharmaceutical ingredient or drug, for example and without limitation, anti-oxidants, buffers, bacteriostats, lipids, liposomes, lipid nanoparticles, emulsifiers, suspending agents, and rheology modifiers. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous solutions and suspensions may be prepared from sterile powders, granules and tablets.

Therapeutic compositions typically must be sterile and stable under the conditions of manufacture and storage. For example, sterile injectable solutions can be prepared by incorporating the active agent in the required amount in an appropriate solvent with one or a combination of ingredients enumerated herein, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, typical methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

Pharmaceutically acceptable salts of any of the compounds described herein also may be used in the methods described herein. Pharmaceutically acceptable salt forms of the compounds described herein may be prepared by conventional methods known in the pharmaceutical arts, and include as a class veterinarily acceptable salts.

For example and without limitation, where a compound comprises a carboxylic acid group, a suitable salt thereof may be formed by reacting the compound with an appropriate base to provide the corresponding base addition salt. Non-limiting examples include: alkali metal hydroxides, such as potassium hydroxide, sodium hydroxide and lithium hydroxide; alkaline earth metal hydroxides, such as barium hydroxide and calcium hydroxide; alkali metal alkoxides, such as potassium ethanolate and sodium propanolate; and various organic bases such as piperidine, diethanolamine, and N-methylglutamine.

Non-limiting examples of pharmaceutically-acceptable base salts include: aluminum, ammonium, calcium, copper, ferric, ferrous, lithium, magnesium, manganic, manganous, potassium, sodium, and zinc salts. Salts derived from pharmaceutically acceptable organic non-toxic bases include, without limitation: salts of primary, secondary, and tertiary amines, substituted amines including naturally occurring substituted amines, cyclic amines, and basic ion exchange resins, such as arginine, betaine, caffeine, chloroprocaine, choline, N,N′-dibenzylethylenediamine (benzathine), dicyclohexylamine, diethanolamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, iso-propylamine, lidocaine, lysine, meglumine, N-methyl-D-glucamine, morpholine, piperazine, piperidine, polyamine resins, procaine, purines, theobromine, triethanolamine, triethylamine, trimethylamine, tripropylamine, and tris-(hydroxymethyl)-methylamine (tromethamine).

Non-limiting examples of pharmaceutically-acceptable acid salts include: acetate, adipate, alginate, arginate, aspartate, benzoate, besylate (benzenesulfonate), bisulfate, bisulfite, bromide, butyrate, camphorate, camphorsulfonate, caprylate, chloride, chlorobenzoate, citrate, cyclopentanepropionate, digluconate, dihydrogenphosphate, dinitrobenzoate, dodecylsulfate, ethanesulfonate, fumarate, galacterate, galacturonate, glucoheptanoate, gluconate, glutamate, glycerophosphate, hemisuccinate, hemisulfate, heptanoate, hexanoate, hippurate, hydrochloride, hydrobromide, hydroiodide, 2-hydroxyethanesulfonate, iodide, isethionate, iso-butyrate, lactate, lactobionate, malate, maleate, malonate, mandelate, metaphosphate, methanesulfonate, methylbenzoate, monohydrogenphosphate, 2-naphthalenesulfonate, nicotinate, nitrate, oxalate, oleate, pamoate, pectinate, persulfate, phenylacetate, 3-phenylpropionate, phosphate, phosphonate, and phthalate.

Multiple salts forms are also considered to be pharmaceutically-acceptable salts. Common, non-limiting examples of multiple salt forms include: bitartrate, diacetate, difumarate, dimeglumine, diphosphate, disodium, and trihydrochloride.

As such, “pharmaceutically acceptable salt” as used herein is intended to mean an active ingredient (drug) comprising a salt form of any compound as described herein. The salt form may confer improved and/or desirable pharmacokinetic/pharmodynamic properties of the compounds described herein.

A “therapeutically effective amount” refers to an amount of a drug product or active agent effective, at dosages and for periods of time necessary, to achieve the desired therapeutic result. An “amount effective” for treatment of a condition is an amount of an active agent or dosage form, such as a single dose or multiple doses, effective to achieve a determinable end-point. The “amount effective” is preferably safe—at least to the extent the benefits of treatment outweighs the detriments, and/or the detriments are acceptable to one of ordinary skill and/or to an appropriate regulatory agency, such as the U.S. Food and Drug Administration. A therapeutically effective amount of an active agent may vary according to factors such as the disease state, age, sex, and weight of the individual, and the ability of the active agent to elicit a desired response in the individual. A “prophylactically effective amount” refers to an amount effective, at dosages and for periods of time necessary, to achieve a desired prophylactic result. Typically, since a prophylactic dose is used in subjects prior to or at an earlier stage of disease, the prophylactically effective amount may be less than the therapeutically effective amount.

Dosage regimens may be adjusted to provide the optimum desired response (e.g., a therapeutic or prophylactic response). For example, a single dose or bolus may be administered, several divided doses may be administered over time, or the composition may be administered continuously or in a pulsed fashion with doses or partial doses being administered at regular intervals, for example, every 10, 15, 20, 30, 45, 60, 90, or 120 minutes, every 2 through 12 hours daily, or every other day, etc., be proportionally reduced or increased as indicated by the exigencies of the therapeutic situation. In some instances, it may be especially advantageous to formulate compositions in dosage unit form for ease of administration and uniformity of dosage. The specification for the dosage unit forms are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

An amount effective to control jet lag, modify chronotype, and/or or modulate a circadian clock of a patient, e.g., a human patient, may be 1 μg to 10 g, or from 1 ng to 100 mg/kg of compound 958 per day, for example an amount to produce 20 μM±99%, 90%, 80%, 70%, 60%, 50%, 40%, 30%, 25%, 20%, 15%, 10%, 0.5%, 1%, e.g., from 1 to 40 μM, or any increment therebetween of the compound in a patient's bodily fluid, e.g., blood, serum, plasma, etc.

The compositions and methods described herein are useful to control jet lag, modify chronotype, and/or or modulate a circadian clock of a patient, e.g., a human patient. “Jet lag” or “jet lag disorder” refers to a temporary sleep disorder resulting from quick traversal of one or more time zones, such that the patient's internal clock or circadian rhythm are disrupted because the patient's internal clock is synced to their original time zone and has not yet changed to the time zone of where the patient has traveled. The more time zones crossed, the more likely a patient will suffer from jet lag, and the longer it will last. Jet lag can cause daytime fatigue, cognitive difficulties, an unwell feeling, trouble staying alert, and stomach problems. Although the symptoms are temporary, they can affect a patient's comfort while on vacation or during a business trip. The more often a patient travels between time zones the greater that patient can suffer from jet lag. Dehydration and airline cabin pressure may also contribute to jet lag. Age also can contribute to jet lag, with older patients often needing more time to recover from jet lag.

“Chronotype” or “diurnal preference” is a part of circadian rhythmicity and may be defined as individual variation in the preferred timing of the sleep-wake cycle. It reflects individual variability in the phase of entrainment and is associated with variations of physiological nature, such as the rhythm of body temperature and hormone secretion. Chronotype may evaluated using self-reported questionnaires, usually the Morningness-Eveningness Questionnaire (MEQ), its reduced five-item version (rMEQ), and the Munich Chronotype Questionnaire (MCTQ). The Composite Scale of Morningness (CSM), the Diurnal Type Scale (DTS), the Circadian Type Questionnaire (CTQ), and the Preference Scale (PS) can also be considered instruments for evaluating chronotype. Other tools that may be used to evaluate chronotypes, include, without limitation, core body temperature, dim light melatonin onset (DLMO), a sleep diary, wrist accelerometry, and actigraphs. Morning chronotype individuals, also known as early chronotypes or larks, prefer to be active in the morning and sleep and wake early. Evening chronotypes, also called late chronotypes or owls, prefer to be active in the evening and sleep and wake up late.

The intermediate chronotype (neutral or neither type) has no preference for morning or evening. Morning chronotype individuals achieve peak physical and mental performance in the early part of the day after waking up. Conversely, evening chronotype individuals have the best mental and physical performance before sleeping (Zou H, et al. Chronotype, circadian rhythm, and psychiatric disorders: Recent evidence and potential mechanisms. Front Neurosci. 2022 Aug. 10; 16:811771).

Vasopressin receptor 1A is a receptor for arginine vasopressin, and is broadly-described (see, e.g., NCBI Gene ID: 552, AVPR1A arginine vasopressin receptor 1A [Homo sapiens (human)], and exemplary protein sequence; NCBI Reference Sequence: NP_000697.1 and UniProt P37288 (V1AR_HUMAN)). Melanopsin is encoded by the gene OPN4 in humans, and also may be referred to as Opsin-4 (see, e.g., NCBI Gene ID: 94233, OPN4 opsin 4 [Homo sapiens (human)], and exemplary protein sequence; NCBI Reference Sequence: NP_001025186.1 and UniProt Q9UHM6 (OPN4_HUMAN)). The sequences described in the preceding are merely exemplary, and the terms “vasopressin 1A receptor” and “melanopsin” embrace sequence variants thereof, including alleles, mutants, variants, isoforms, polymorphs, and equivalent proteins from non-human species.

As described above, provided herein are methods of reducing or controlling jet lag, modifying chronotype, and/or modulating a circadian clock of a patient, e.g., a human patient, comprising administering a vasopressin 1A receptor antagonist to the patient in an amount effective to reduce or control jet lag, modify chronotype, and/or modulate a circadian clock of the patient; and/or administering a melanopsin agonist to the patient in an amount effective to reduce or control jet lag, modify chronotype, and/or or modulate a circadian clock of the patient.

Also provided are compositions comprising an active agent, that is a vasopressin 1A receptor antagonist and/or a melanopsin agonist as described herein, comprising the active agent and a pharmaceutically-acceptable excipient, where the active agent is present in an amount effective to reduce or control jet lag, modify chronotype, and/or modulate a circadian clock of a patient. The composition may be a topical, ocular solution, e.g., eyedrops, in the case of a melanopsin agonist. In the case of any of the vasopressin 1A receptor antagonists and/or a melanopsin agonists as described herein, the composition may be an intravenous, transdermal, transmucosal, or oral dosage form, comprising the active agent with suitable excipients to form a useful dosage form for delivery to the active agent compound.

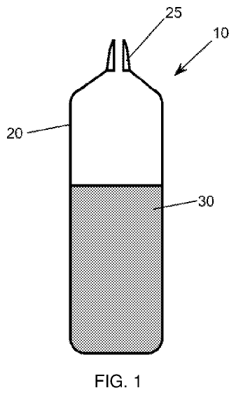

A melanopsin agonist may best be delivered topically to the eye (that is ocularly). The melanopsin agonist may be provided in a drop-dispensing device, such as an eyedrop-dispensing bottle, as are broadly-known in the pharmaceutical arts. For example, the melanopsin agonist may be provided in a plastic dropper bottle as is common for eye drop-delivered drug products. As a non-limiting, illustrative example FIG. 1 depicts a dropper bottle 10 in schematic cross section with a reservoir portion 20 and a dropper tip 25, and containing a liquid drug product composition 30 containing a melanopsin agonist, as described herein. Suitable liquid compositions for ocular delivery include the described drug product as active agent as well as, for example and without limitation: water, rheology modifiers, preservatives, salts, buffers, emulsifiers, and penetration enhancers. A person of ordinary skill in the pharmaceutical and compounding arts can readily ascertain an optimal composition for ocular, oral, or parenteral delivery of the compounds as described herein. An exemplary concentration range for the compounds described herein for an ocular liquid may be from 1 μg/mL to 100 mg/mL, including any effective increment therebetween and a single dose may be from one to five drops in each eye per dose.

A vasopressin 1A receptor antagonist may be administered to reduce or control jet lag, modify chronotype, and/or modulate a circadian clock of a patient, such as a human patient. Non-limiting examples of vasopressin 1A receptor antagonists include: ZINC257318833, MolPort-010-769-806, MolPort-010-769-803, MolPort-004-227-511, MolPort-009-703-593, MolPort-009-703-587, MolPort-003-361-612, MolPort-000-810-610, MolPort-019-855-958, ZINC100959508, MolPort-007-684-659, MolPort-007-880-661, MolPort-002-641-911, MolPort-010-799-948, MolPort-003-073-533, MolPort-002-260-938, MolPort-006-539-702, ZINC24843601, ZINC20611382, ZINC16845443, ZINC10233808, ZINC20611384, ZINC20610887, ZINC62728312, ZINC12231390, or a pharmaceutically acceptable salt thereof.

The vasopressin 1A receptor antagonist may be a compound illustrated in the following structure (i):

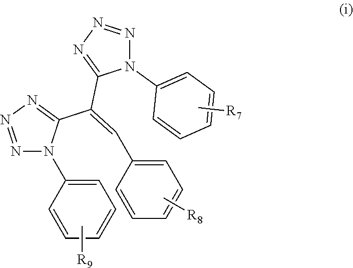

wherein R₇, R₈, and R₉ may be, independently, H, hydroxyl, methyl, or nitro, and in one example, at least one of R₇, R₈, and R₉ may be OH, methyl, or nitro, or a pharmaceutically-acceptable salt thereof.

The vasopressin 1A receptor antagonist may be a compound having the structure (ii):

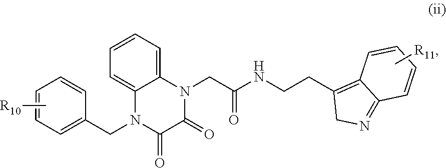

wherein R₁₀ and R₁₁ may be, independently: H, hydroxyl, or methyl, or a pharmaceutically-acceptable salt thereof. In one example, the compound may have the following structure (ii, e.g., MolPort-007-880-661):

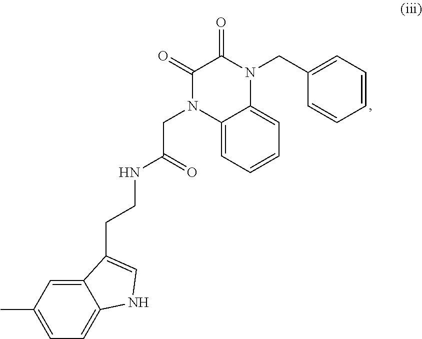 [SMILES: Cc1ccc2[nH]cc(CCNC(=O)Cn3c(=O)c(=O)n(Cc4ccccc4)c4ccccc43)c2c1]

or a pharmaceutically-acceptable salt thereof.

The compound may exclude MolPort-007-880-661, MolPort-003-073-533, and/or MolPort-002-260-938.

The vasopressin 1A receptor antagonist described above may be delivered by any effective route, but may preferably be delivered systemically, e.g., via oral, parenteral, or transdermal routes, such as, for example and without limitation, intramuscularly, subdermally, intravenously, enterically, or trans-mucosally. As above, an ordinary practitioner in the pharmaceutical arts can formulate effective dosage forms for delivery of the vasopressin 1A receptor antagonist.

A melanopsin agonist may be administered to reduce or control jet lag, modify chronotype, and/or modulate a circadian clock of a patient, such as a human patient. Non-limiting examples of melanopsin agonists include: MolPort-046-192-342, MolPort-046-751-265, MolPort-027-820-910, MolPort-039-341-893, MolPort-000-786-730, MolPort-004-901-380, MolPort-002-739-585, MolPort-005-950-241, MolPort-047-468-801, MolPort-005-950-837, MolPort-002-983-412, MolPort-047-768-812, MolPort-002-120-903, and MolPort-000-525-975, ZINC91651216, ZINC270133816, ZINC585287286, ZIN0000334163086, ZIN0000027795402, ZIN0000000974767, ZINC000016952484, ZINC000002762284, ZINC000009487830, ZINC000034034721, and ZINC000015972050, or a pharmaceutically acceptable salt thereof.

The melanopsin agonist may be a compound illustrated in the following structure (iv):

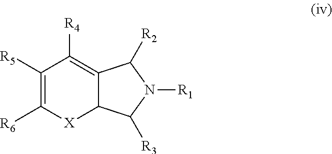

where,
R₁ may be an aromatic five-membered heterocycle group, optionally linked by a methylene group, comprising one or both of N and O, such as furanyl and oxazolyl, including 1,2-oxazolyl and 1,3-oxazolyl, as well as methylfuranyl and methyloxazolyl, including methyl-1,2-oxazolyl and methyl-1,3-oxazolyl,
R₂ may be oxygen (carbonyl), phenyl, or halo-substituted phenyl, such as o, m, or p-fluoro phenyl,
R₃ may be oxygen (carbonyl) or indolyl,
R₄ may be H or oxygen (carbonyl),
R₅ and R₆ may be both H, or together form a phenyl or naphthyl group, and
X may be C or O.

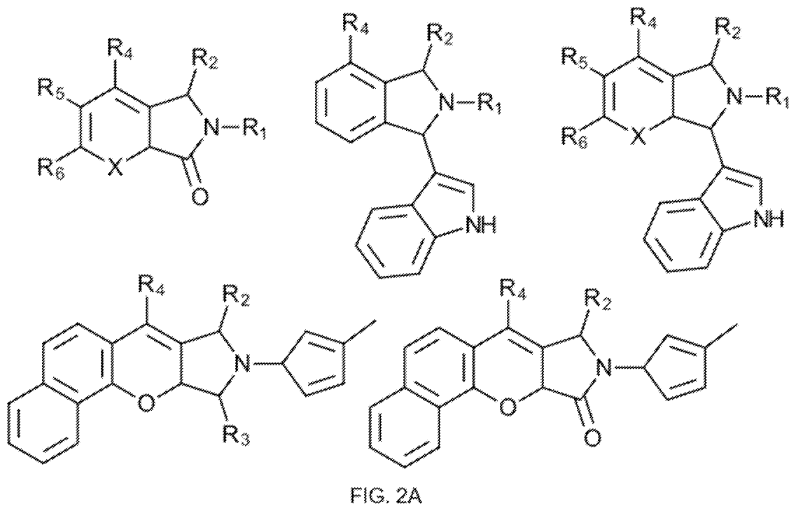

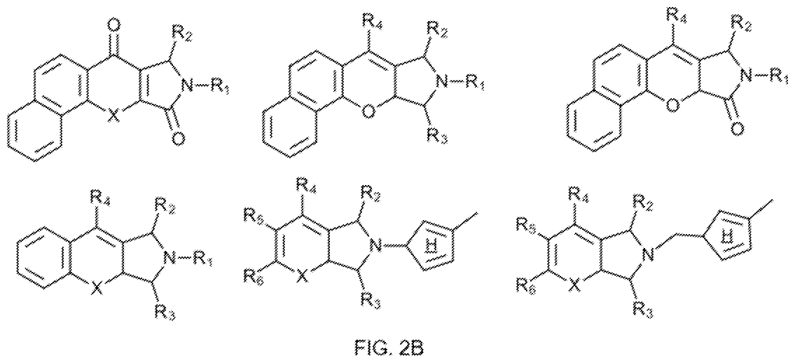

The melanopsin agonist may exclude MolPort-002-983-412 and/or MolPort-000-525-975. Non-limiting examples of such compounds include those variants of structure (iii) depicted in FIGS. 2A and 2B.

The melanopsin agonist described above may be delivered by any effective route, but may preferably be delivered topically as an eye drop, e.g., contained within and dispensed by a dropper bottle, for example as depicted in FIG. 1.

As above, an ordinary practitioner in the pharmaceutical arts can readily formulate effective dosage forms for delivery of the melanopsin agonist and vasopressin 1A receptor antagonist, including pharmaceutically-acceptable salts thereof, as described herein.

### EXAMPLES

Activation of melanopsin and the vasopressin receptor in the retina and downstream neural circuitry connecting the eye to the suprachiasmatic nucleus (SCN) in the brain are involved in the circadian control of jet lag. A computational approach was pursued to identify allosteric activators of these two receptors. This entailed druggability simulations using probe compounds that contain drug-like functional groups and/or features shared by the lead compounds. The most important binding pockets were identified with evaluation of binding poses of probes, and pharmacophore modeling, using Pharmmaker. Virtual screening was performed of pharmacophore models against libraries of small compounds which are docked onto the target using the Pharmit server. A ranked list of small molecule compounds predicted to bind and activate these receptors was generated. Described herein, including in the examples below, is the use of specific small molecules to activate melanopsin and/or the vasopressin receptor to control or reduce jet lag, modify chronotype, and/or or modulate a circadian clock of a patient, e.g., a human patient.

### Example 1—Vasopressin Receptor

Arginine vasopressin receptor (AVPR1a or vasopressin receptor) is a receptor for vasopressin, a neurohormone synthesized in the hypothalamus and secreted into the blood by the pituitary gland. Vasopressin has been shown to play a central role in regulating blood pressure, body-fluid balance, and normal cellular functions. Light-induced vasopressin release enhances the responses of SCN neurons to light. It also enhances expression of genes involved in photo-entrainment of biological rhythms. AVPR1a antagonists thus interfere with the signal strength and thereby can bring about entrainment. Mice genetically deficient in vasopressin V1a and V1 b receptors are resistant to jet lag. We are identifying and testing compounds that block this receptor. Unlike a melanopsin activator as described in Example 1, that may be preferably delivered directly to the eye, an AVPR1a receptor drug may be preferably given systemically.

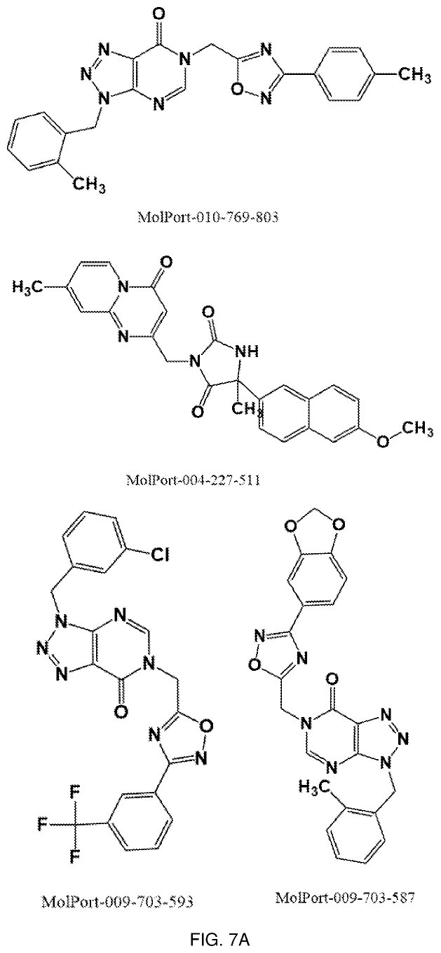

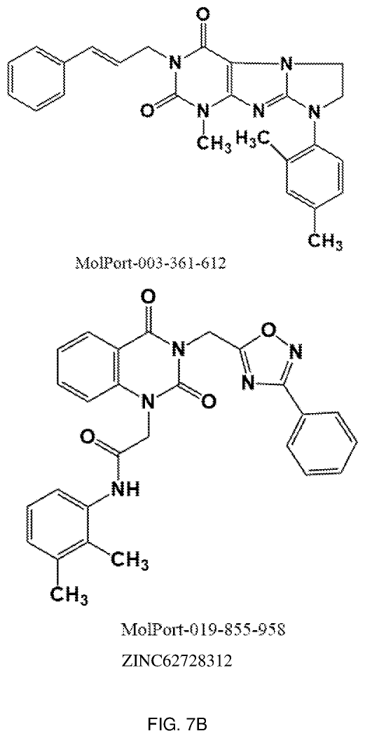

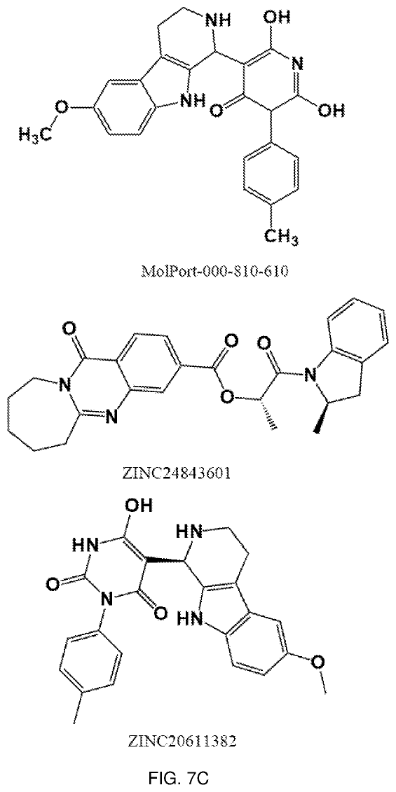

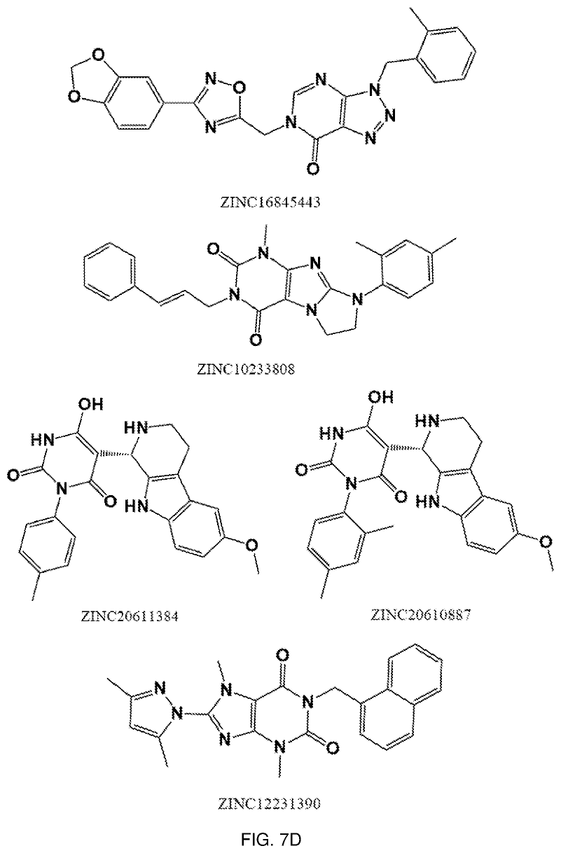

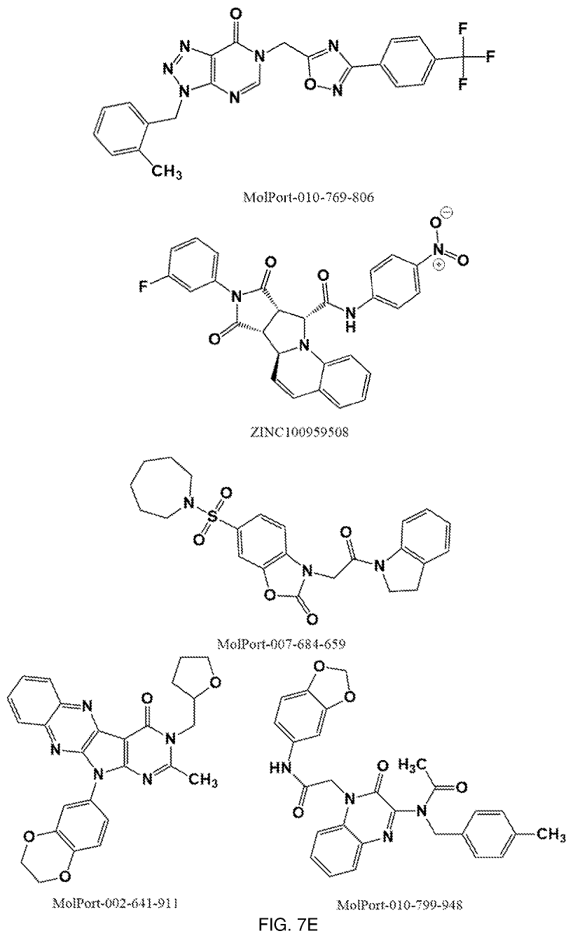

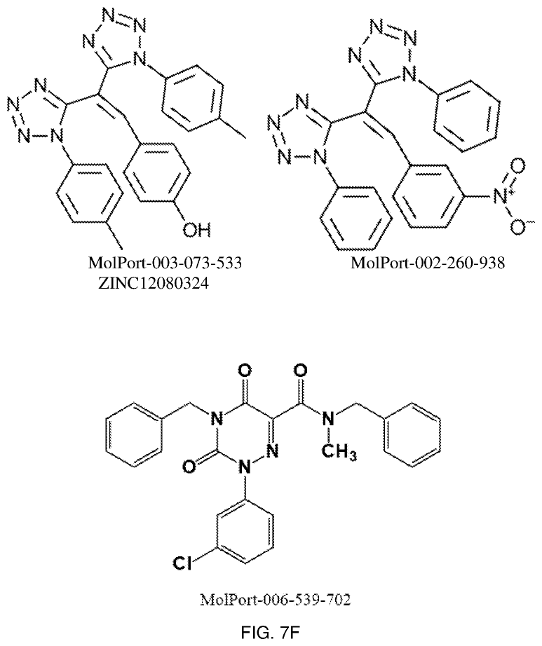

The following supports the efficacy of this treatment mode. Initial screening details are provided in priority U.S. Provisional Patent Application No. 63/374,459 filed Sep. 2, 2022, the disclosure of which is hereby incorporated herein by reference in its entirety. The following compounds were identified in initial screenings as having potential for targeting the AVPR1a receptor for use in controlling jet lag: ZINC257318833, MolPort-010-769-806, MolPort-010-769-803, MolPort-004-227-511, MolPort-009-703-593, MolPort-009-703-587, MolPort-003-361-612, MolPort-000-810-610, MolPort-019-855-958, ZINC100959508, MolPort-007-684-659, MolPort-007-880-661, MolPort-002-641-911, MolPort-010-799-948, MolPort-003-073-533, MolPort-002-260-938, MolPort-006-539-702, ZINC24843601, ZINC20611382, ZINC16845443, ZINC10233808, ZINC20611384, ZINC20610887, ZINC62728312, ZINC12231390. Further analysis, as described below, identified 533 (MolPort-003-073-533), 938 (MolPort-002-260-938), and compound 661 (MolPort-007-880-661) as leading candidates. FIGS. 7A-7F provide exemplary structures for the MolPort and ZINC compounds.

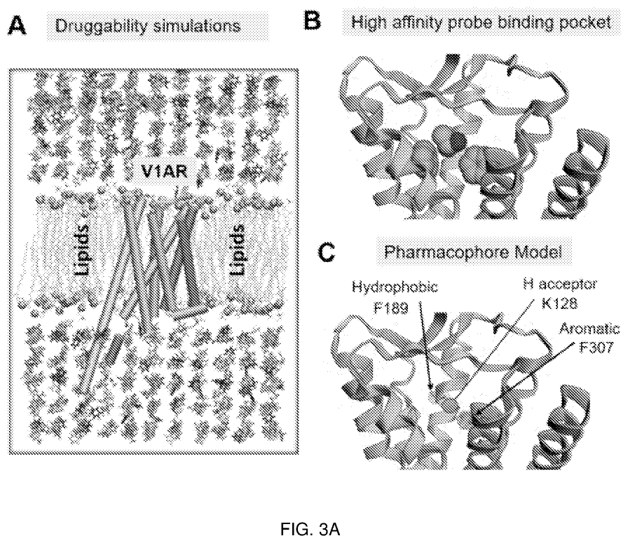

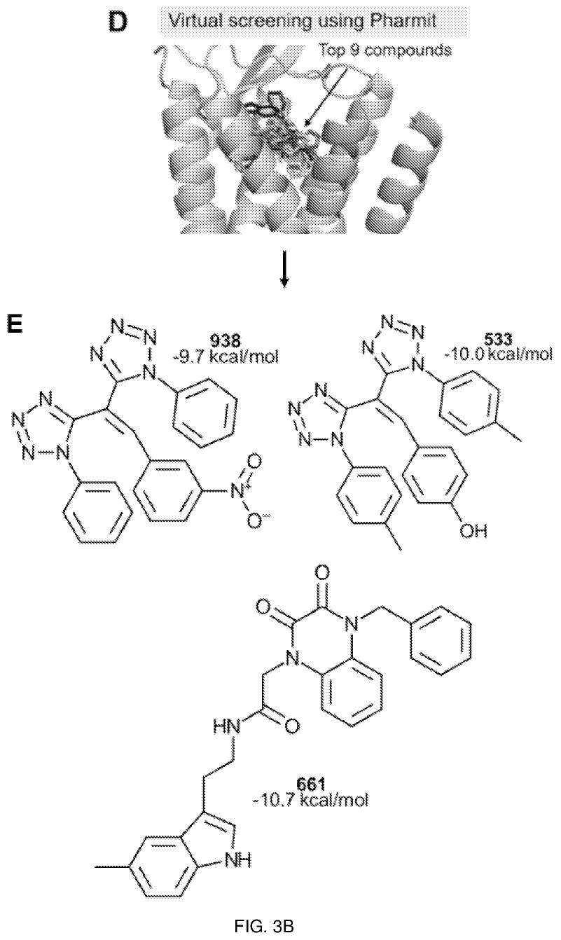

In our pursuit of in silico discovery of vasopressin 1a receptor (V1AR) antagonists, we performed a series of structure-based computations including three components: druggability simulations, pharmacophore modeling and virtual screening (FIG. 3A). AlphaFold2-modeled human V1AR was embedded into membrane lipids and solvated in solutions containing drug-like probe molecules. We added seven different probe molecules: acetamide, acetate, benzene, imidazole, isobutane, isopropanol, isopropylamine, and 4-H pyran. An initial configuration of our druggability simulation is showed in FIG. 3A. Using our tool Pharmmaker, we constructed a pharmacophore model, and selected the high affinity residues (F189, K128, and F307) at the molecular pocket identified as the target site. Three features were used to generate our pharmacophore model: hydrophobic feature (near F189), aromatic feature (near F307), and an H acceptor (near K138). We screened compounds from MolPort using Pharmit. Among the predicted top 9 compounds, three were validated by in vitro experiments (FIG. 3B). Those three effective V1AR inhibitors are sub-grouped into two based on molecular structure similarity as shown below: one including compound 661 (MolPort-007-880-661), and the other contains compound 533 (MolPort-003-073-533) and 938 (MolPort-002-260-938).

Structure i depicts generically a first embodiment of an AVPR1a receptor antagonist that includes compounds 938 and 533:

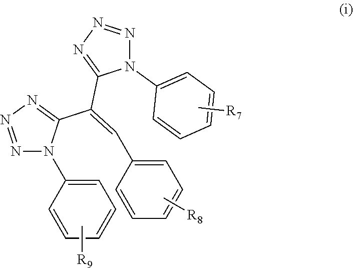

wherein R₇, R₈, and R₉ may be, independently, H, OH, methyl, or nitro, and in one example, at least one of R₇, R₈, and R₉ may be OH, methyl, or nitro.

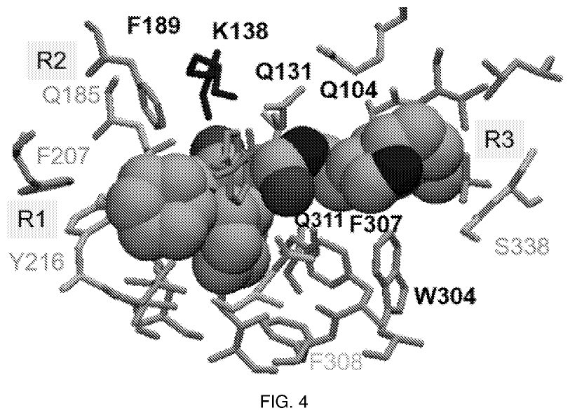

To identify the generic core structure of compound 661, multiple all-atomic molecular dynamics (MD) simulations within explicit membrane lipids were performed. MD simulations confirmed high affinity binding of compound 661 (−10.5±0.5 kcal/mol) during the entire simulation courses. Moreover, MD simulations revealed the functional groups engaged into the binding of compound 661 to V1AR. Of note, the three ketones from compound 661 formed stable hydrogen bonds with Q104/Q131, K138, and Q311 from V1AR. Besides, aromatic interactions with F189, F307/F308 and W304 contributed significantly to the binding of compound 661 (FIG. 4). Conducting MD simulations on the binding of compound 661 to V1AR allowed us to deduce the generic structure of compound-661-like analogues having the structure (ii):

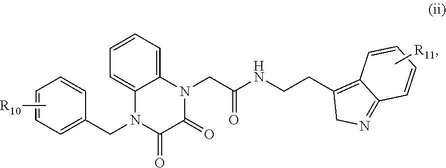

wherein R₁₀ and R₁₁ may be, independently: H, hydroxyl, or methyl.

### Example 2—Melanopsin

Melanopsin is a G-protein coupled receptor expressed in the retina of the eye and is involved in conveying light exposure to the SCN to reset the master clock. It responds most strongly to blue light to project information through the retinohypothalamic tract to the suprachiasmatic nucleus (SCN) in the hypothalamus, which synchronizes internal biological and psychological rhythms. The melanopsin receptor is an excellent target for treating or preventing jet lag as it is not involved in normal visual processing, and manipulation of the function of this receptor should not impact vision. Moreover, given its function specifically in the eye, the drug may be preferably deposited topically in an eye drop solution, reducing any potential side effects that could result from taking oral medication.

The following supports the efficacy of this treatment mode. Initial screening details are provided in priority U.S. Provisional Patent Application No. 63/374,459 filed Sep. 2, 2022, the disclosure of which is hereby incorporated herein by reference in its entirety. The following compounds were identified in initial screenings as having potential for targeting melanopsin for use in controlling jet lag: MolPort-046-192-342, MolPort-046-751-265, MolPort-027-820-910, MolPort-039-341-893, MolPort-000-786-730, MolPort-004-901-380, MolPort-002-739-585, MolPort-005-950-241, MolPort-047-468-801, MolPort-005-950-837, MolPort-002-983-412, MolPort-047-768-812, MolPort 002-120-903, MolPort-000-525-975, ZINC91651216, ZINC270133816, ZINC585287286, ZINC000334163086, ZINC000027795402, ZINC000000974767, ZINC000016952484, ZINC000002762284, ZINC000009487830, ZINC000034034721, and ZINC000015972050. Further analysis, as described below, identified MolPort-002-983-412 and MolPort-000-525-975 as leading candidates. FIGS. 7A-7F provide exemplary structures for the MolPort and ZINC compounds.

Toward in silico discovery of a small molecule modulator of melanopsin, we performed structure-based computations composed of three parts: druggability simulations, pharmacophore modeling, and virtual screening. Druggability simulations were carried out using the model structure of melanopsin in the presence of explicit water and probe molecules representative of drug-like fragments. Acetamide, acetate, benzene, imidazole, isobutane, isopropanol, and isopropylamine were used as probe molecules in six independent runs of 40 ns each. Using our tool Pharmmaker, we constructed a pharmacophore model, and selected the high affinity residues (H70, L73, L217, and S333) at the molecular pocket identified as the target site. The interactions of these residues with the probe molecules were ranked based on their occurrence frequency during simulations. Notably, among the probe molecules, three benzenes demonstrated a high propensity to engage in close interactions with melanopsin residues.

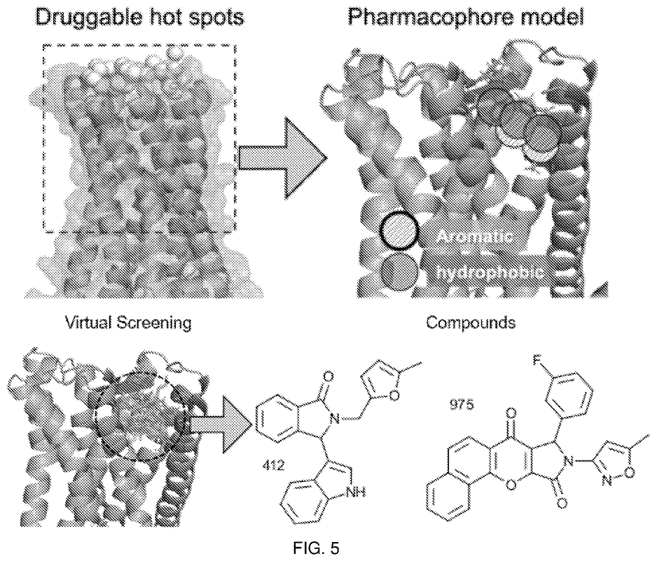

Molecular dynamics snapshots that simultaneously displayed multiple frequently observed (that is, entropically favored) interactions were used to construct a pharmacophore model (FIG. 5). The pharmacophore model was composed of three hydrophobic rings and two aromatic rings. It was then screened against the ZINC and MolPort small molecule libraries using the Pharmit server to obtain an ensemble of compounds. The top scoring compounds were selected as hits for further experimental validation. Experimental tests suggested that compounds 975 (MolPort-000-525-975) and 412 (MolPort-002-983-412) could be adopted as lead compounds (activators). The binding behavior of the compounds was then examined through molecular dynamics simulations and characterize the key interactions that enable their activity.

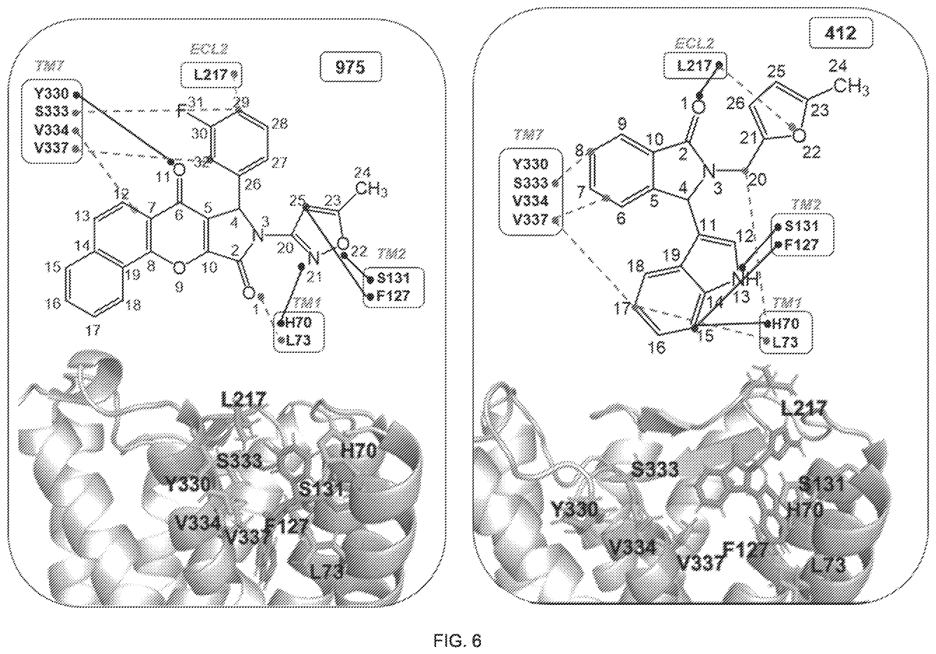

FIG. 6 depicts two lead compounds, 975 and 412 and melanopsin functional groups or residues that are involved in target protein (ribbon diagram)—drug (stick) interactions.

Refinement of the identified compounds 975 and 412 led to the prediction of binding properties. All-atom 1 μs molecular dynamics simulations (five independent runs of 0.2 μs each) with explicit membrane conducted for each compound were analyzed to characterize the most critical functional groups and interactions. Seven residues—H70, L73, F127, S131, L217, S333, and V337—exhibit interactions with both compounds (see FIG. 6). Three of them—H70, F127, and S131—have strong interactions in both cases. The residue S131 interacts with the N or O atoms of the drugs in both cases, and S333/V337 interacts with a 6-membered ring. The two compounds share the core group illustrated in the following structure (iv):

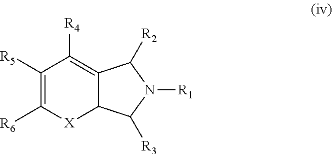

where,
R₁ may be an aromatic five-membered heterocycle group, optionally linked by a methylene group, comprising one or both of N and O, such as furanyl and oxazolyl, including 1,2-oxazolyl and 1,3-oxazolyl, as well as methylfuranyl and methyloxazolyl, including methyl-1,2-oxazolyl and methyl-1,3-oxazolyl,
R₂ may be oxygen (carbonyl), phenyl, or halo-substituted phenyl, such as o, m, or p-fluoro phenyl,
R₃ may be oxygen (carbonyl) or indolyl,
R₄ may be H or oxygen (carbonyl),
R₅ and R₆ may be both H, or together form a phenyl or naphthyl group, and
X may be C or O.

Variations of the structure of structure iv are depicted in FIGS. 2A and 2B.

### Example 3—Compound Screening

Presto-Tango Assay: Empty Tango plasmid was generated from Addgene plasmid #66225. Melanopsin was amplified by pcr from pAAV-GFAP104-melanopsin-mCherry (Addgene plasmid #122630) and AVPR1a from hV1aR-GFP (Addgene plasmid #67846) and cloned to generate OPN4- and AVPR1a-Tango plasmid. HTLA cells (a HEK293 cell line stably expressing a tTA-dependent luciferase reporter and a β-arrestin2-TEV fusion gene—a gift from Bryan Roth) was transfected with OPN4-Tango or AVPR_{1A}-Tango plasmids. The HTLA Cells are grown in DMEM supplemented with 10% FBS, 100 U/ml penicillin and 100 μg/ml streptomycin, 2 μg/ml puromycin and 100 μg/ml hygromycin B (HTLA medium) in a humidified atmosphere at 37 °C in 5% CO₂ (HTLA Medium).

The receptors were activated using Fluorescent LED light and 9-cis retinal (Sigma-R5744) for OPN4 and argipressin (Cayman Chemicals-24154) for AVPR_{1A}. β-arrestin-TEV protease recruited to V2 tail results in TEV-cleavage to release the tetracycline transactivator tTA to trans-locate to the Nucleus and tTA dependant Luciferase expression. Luciferase activity was measured using Bright-Glo Luciferase (Promega #E2160) assay. 9-cis-Retinol showed an EC50 of ~250 nM and argipressin ~1.2 nM. Based on this, compounds were screened at 0.5 and 1.0 μM 9cis-retinol and 5.0 and 10 nM argipressin concentrations.

Day 1: HTLA cells were plated in at 9 to 10×10⁶ cells per 100 mm cell culture dish in HTLA Medium.

Day 2: The cells were transfected using the Polyethylenimine method.

Day 3: The transfected cells were transferred at 150000 to 200000 cells per well in 100 μL of medium into poly-D-lysine coated and rinsed 96-well white clear-bottom cell culture plates.

Day 4: A serial dilution of respective compounds ranging from 10 μM-100 μM were prepared in the HTLA medium along with 5 and 10 μM 9cis-retinal. The cells were illuminated using a LED light fixture within a CO₂ incubator providing a humidified atmosphere at 37 °C in 5% CO₂ for 16 hours. 1 nM Argipressin (Medchem Express) was used in place of 9cis-retinal for the AVPR1a assays without illumination for 16 hrs.

Day 5: The medium containing drug solutions were removed from the wells (by aspiration or shaking), and 50 μL per well of Bright-Glo solution (Promega) diluted 10-fold in filter-sterilized assay buffer, which consisted of 20 mM HEPES and 1×HBSS at pH 7.4 and 1 mM Glucose were added to each well. After incubation for 15 to 20 minutes at room temperature, luminescence was counted in a BioTek-Flx-800 luminescence counter. Results in the form of luminescence units were exported into Excel spreadsheets, and Graphpad Prism was used for data analysis.

The Ratio of luminescence units obtained in presence of the compounds to the positive control was plotted against log [compound-M] in molarity and fitted using regression analysis.

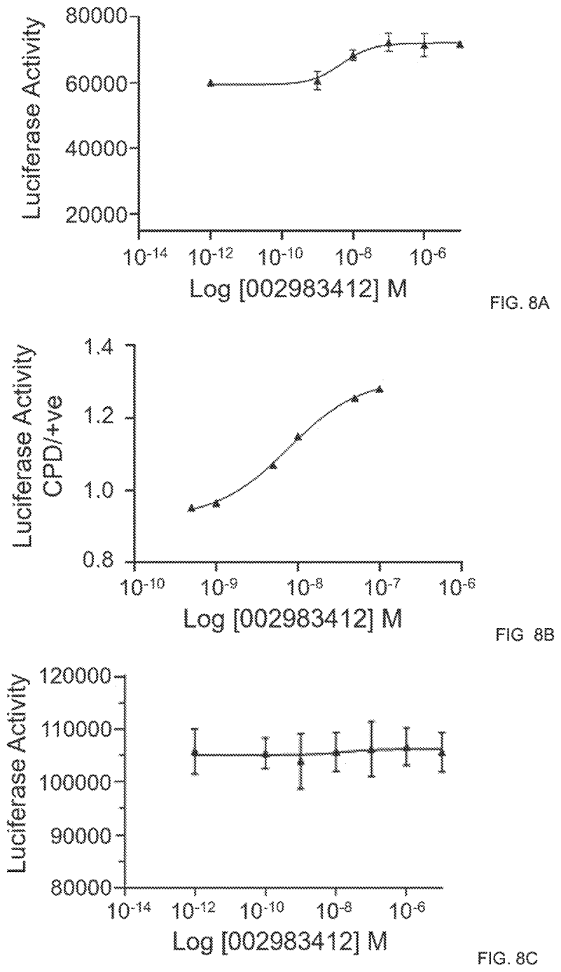

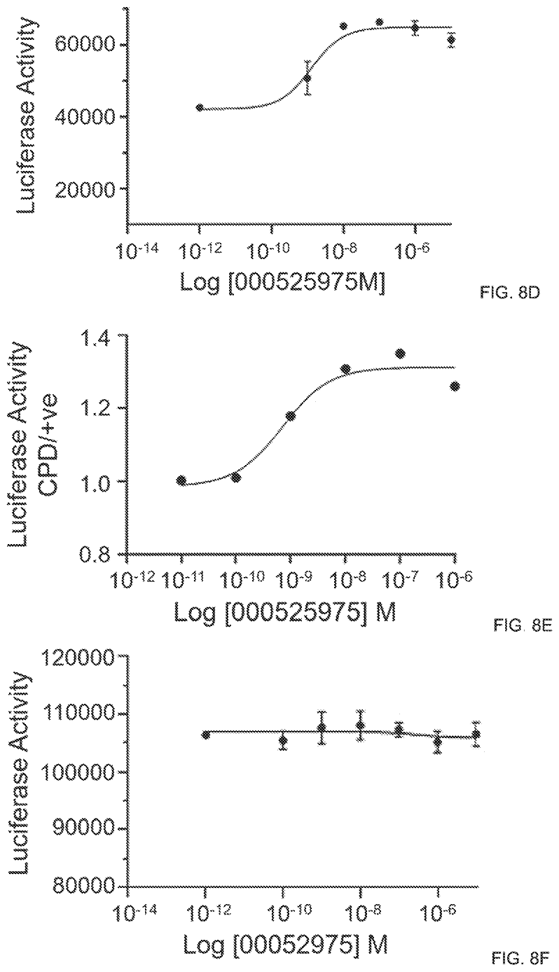

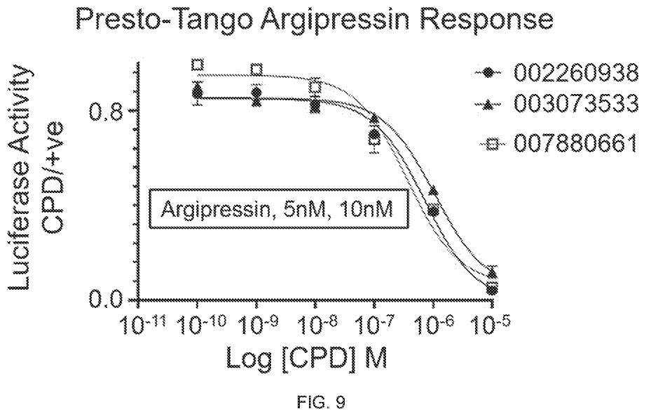

Melanopsin Screen: Eighteen compounds identified as allosteric activators by computational analysis were procured from Molport. They were resuspended in 100% DMSO at 10 mM concentrations as stock. Serial dilutions going from 10 μM-100 μM diluted in HTLA medium were assayed to identify Melanopsin allosteric activator. Of the 18 molecules tested we identified two as strong activators of Melanopsin at an EC50 values of ~7.5 nM and ~0.87 nM (see, FIGS. 8A-8F). AVPR1a Screen: Twelve compounds identified as allosteric inhibitors by computational analysis were procured from MolPort. They were resuspended in 100% DMSO at 10 mM concentrations as stock. Serial dilutions going from 10 μM-1 nM, diluted in HTLA medium, were assayed to identify the allosteric inhibitors of AVPR1a. Of the 12 molecules, till now, we have tested 10 against AVPR1a using the Presto-Tango assay, with argipressin as the GPCR activating Ligand. We have so far been able to identify one strong activator (EC50-~2.0 μM), three strong inhibitors (EC50 ~265 nM, ~600 nM, ~1.0 μM) and two weak inhibitors (EC50 ~81 nM and ~500 nM) (see, FIG. 9). The weak inhibitors were only 50% efficient (to DMSO controls) most probably due to the compounds tested being susceptible to enzymatic degradation from the HTLA cells.

It will be readily appreciated by those skilled in the art that modifications may be made to the invention without departing from the concepts disclosed in the foregoing description. Accordingly, the particular embodiments described in detail herein are illustrative only and are not limiting to the scope of the invention, which is to be given the full breadth of the appended claims and any and all equivalents thereof.

1. A method of reducing or controlling jet lag, modifying chronotype, and/or modulating a circadian clock of a patient, comprising:
administering a vasopressin 1A receptor antagonist to the patient in an amount effective to reduce or control jet lag, modify chronotype, and/or modulate a circadian clock of the patient; and/or
administering a melanopsin agonist to the patient in an amount effective to reduce or control jet lag, modify chronotype, and/or modulate a circadian clock of the patient.

2-3. (canceled)

4. The method of claim 1, wherein the vasopressin 1A receptor antagonist is selected from the group consisting of: ZINC257318833, MolPort-010-769-806, MolPort-010-769-803, MolPort-004-227-511, MolPort-009-703-593, MolPort-009-703-587, MolPort-003-361-612, MolPort-000-810-610, MolPort-019-855-958, ZINC100959508, MolPort-007-684-659, MolPort-007-880-661, MolPort-002-641-911, MolPort-010-799-948, MolPort-003-073-533, MolPort-002-260-938, MolPort-006-539-702, ZINC24843601, ZINC20611382, ZINC16845443, ZINC10233808, ZINC20611384, ZINC20610887, ZINC62728312, ZINC12231390, or a pharmaceutically acceptable salt thereof.

5. (canceled)

6. The method of claim 1, wherein the vasopressin 1A receptor antagonist comprises a compound, having the structure (i): 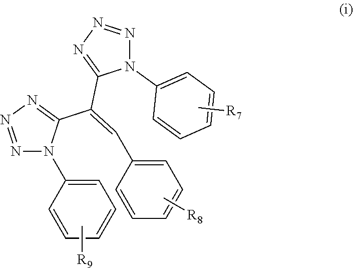
wherein R₇, R₈, and R₉ are, independently, H, hydroxyl, methyl, or nitro, and optionally at least one of R₇, R₈, and R₉ are OH, methyl, or nitro, or a pharmaceutically-acceptable salt thereof.

7. The method of claim 6, wherein R₇ and R₉ are H, and R₈, is nitro, or a pharmaceutically-acceptable salt thereof.

8. The method of claim 1, wherein the vasopressin 1A receptor antagonist comprises a compound, having the structure (ii): 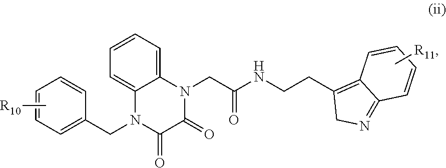
wherein R₁₀ and R₁₁ are, independently: H, hydroxyl, or methyl,
or a pharmaceutically-acceptable salt thereof.

9. The method of claim 1, wherein the vasopressin 1A receptor antagonist comprises a compound (ii): 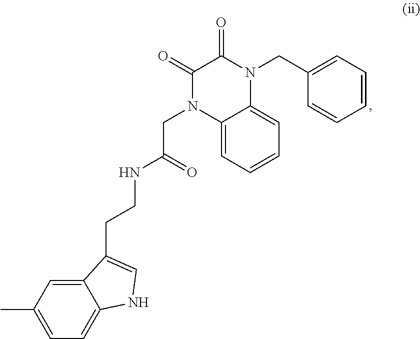 [SMILES: Cc1ccc2[nH]cc(CCNC(=O)Cn3c(=O)c(=O)n(Cc4ccccc4)c4ccccc43)c2c1]
or a pharmaceutically-acceptable salt thereof.

10. (canceled)

11. The method of claim 1, wherein the melanopsin agonist comprises a compound selected from the group consisting of: MolPort-046-192-342, MolPort-046-751-265, MolPort-027-820-910, MolPort-039-341-893, MolPort-000-786-730, MolPort-004-901-380, MolPort-002-739-585, MolPort-005-950-241, MolPort-047-468-801, MolPort-005-950-837, MolPort-047-768-812, MolPort-002-120-903, ZINC91651216, ZINC270133816, ZINC585287286, ZINC000334163086, ZINC000027795402, ZINC000000974767, ZINC000016952484, ZINC000002762284, ZINC000009487830, ZINC000034034721, and ZINCO000015972050, or a pharmaceutically acceptable salt thereof.

12. The method of claim 1, wherein the melanopsin agonist comprises MolPort-002-983-412, MolPort-000-525-975, or a pharmaceutically-acceptable salt thereof.

13. (canceled)

14. The method of claim 1, wherein the melanopsin agonist comprises a compound having the structure (iv): 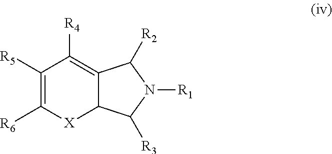
wherein,
R₁ is an aromatic five-membered heterocycle group, optionally linked by a methylene group, comprising one or both of N and O,
R₂ is carbonyl, phenyl, or halo-substituted phenyl,
R₃ is oxygen or indolyl,
R₄ is H or oxygen,
R₅ and R₆ are both H, or together form a phenyl or naphthyl group, and
X is C or O,
or a pharmaceutically-acceptable salt thereof.

15. The method of claim 14, wherein the melanopsin agonist comprises a compound having the structure: 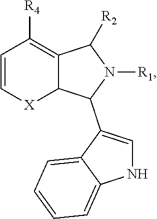
or a pharmaceutically-acceptable salt thereof.

16. The method of claim 14, wherein the melanopsin agonist comprises a compound having the structure: 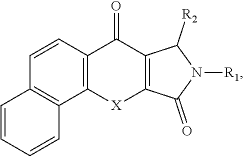
or a pharmaceutically-acceptable salt thereof.

17. The method of claim 1, comprising: administering both a vasopressin 1A receptor antagonist and a melanopsin agonist to the patient, in amounts effective to reduce or control jet lag, modify chronotype, and/or or modulate a circadian clock of the patient.

18. A composition, comprising, a vasopressin 1A receptor antagonist and/or a melanopsin agonist, in amounts effective to reduce or control jet lag, modify chronotype, and/or modulate a circadian clock of the patient, and a pharmaceutically-acceptable excipient.

19. (canceled)

20. The composition of claim 1, wherein the vasopressin 1A receptor agonist is selected from the group consisting of: ZINC257318833, MolPort-010-769-806, MolPort-010-769-803, MolPort-004-227-511, MolPort-009-703-593, MolPort-009-703-587, MolPort-003-361-612, MolPort-000-810-610, MolPort-019-855-958, ZINC100959508, MolPort-007-684-659, MolPort-007-880-661, MolPort-002-641-911, MolPort-010-799-948, MolPort-003-073-533, MolPort-002-260-938, MolPort-006-539-702, ZINC24843601, ZINC20611382, ZINC16845443, ZINC10233808, ZINC20611384, ZINC20610887, ZINC62728312, ZINC12231390, or a pharmaceutically acceptable salt thereof.

21. (canceled)

22. The composition of claim 18, wherein the vasopressin 1A receptor antagonist comprises a compound, having the structure (i): 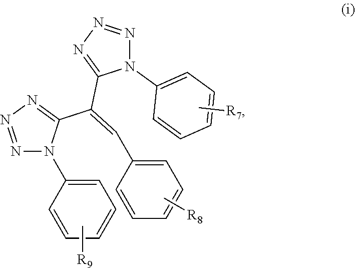
wherein R₇, R₈, and R₉ are, independently, H, hydroxyl, methyl, or nitro, and optionally at least one of R₇, R₈, and R₉ are OH, methyl, or nitro, or a pharmaceutically-acceptable salt thereof, and wherein the vasopressin 1A receptor antagonist does not comprise MolPort-002-260-938 or MolPort-007-880-661.

23. (canceled)

24. The composition of claim 22, wherein R₇ and R₉ are H, and R₈, is nitro, or a pharmaceutically-acceptable salt thereof.

25. The composition of claim 18, comprising a compound having the structure (ii): 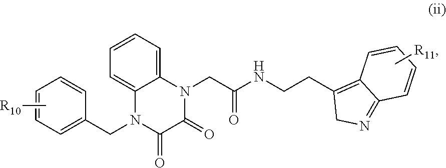
wherein R₁₀ and R₁₁ are, independently: H, hydroxyl, methyl, or a pharmaceutically-acceptable salt thereof.

26. The composition of claim 18, wherein the vasopressin 1A receptor antagonist comprises a compound, having the structure (iii): 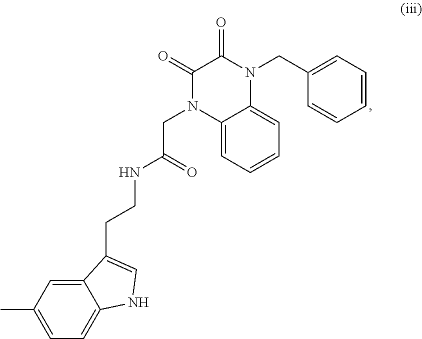 [SMILES: Cc1ccc2[nH]cc(CCNC(=O)Cn3c(=O)c(=O)n(Cc4ccccc4)c4ccccc43)c2c1]
or a pharmaceutically-acceptable salt thereof.

27. (canceled)

28. The composition of claim 18, wherein the melanopsin agonist comprises a compound selected from the group consisting of: MolPort-046-192-342, MolPort-046-751-265, MolPort-027-820-910, MolPort-039-341-893, MolPort-000-786-730, MolPort-004-901-380, MolPort-002-739-585, MolPort-005-950-241, MolPort-047-468-801, MolPort-005-950-837, MolPort-047-768-812, MolPort-002-120-903, ZINC91651216, ZINC270133816, ZINC585287286, ZINC000334163086, ZINC000027795402, ZINC000000974767, ZINC000016952484, ZINC000002762284, ZINC000009487830, ZINC000034034721, and ZINCO000015972050, or a pharmaceutically acceptable salt thereof.

29. The composition of claim 18, wherein the melanopsin agonist comprises MolPort-002-983-412, MolPort-000-525-975, or a pharmaceutically-acceptable salt thereof.

30. (canceled)

31. The composition of claim 18, wherein the melanopsin agonist comprises a compound having the structure (iv): 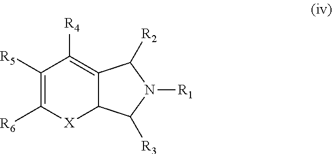
wherein,
R₁ is an aromatic five-membered heterocycle group, optionally linked by a methylene group, comprising one or both of N and O,
R₂ is carbonyl, phenyl, or halo-substituted phenyl,
R₃ is oxygen or indolyl,
R₄ is H or oxygen,
R₅ and R₆ are both H, or together form a phenyl or naphthyl group, and
X is C or O,
wherein the melanopsin agonist does not comprise MolPort-002-983-412 or MolPort-000-525-975,
or a pharmaceutically-acceptable salt thereof.

32. The composition of claim 31, wherein the melanopsin agonist comprises a compound having the structure: 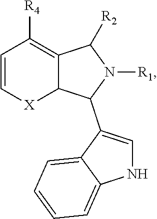
or a pharmaceutically-acceptable salt thereof.

33. The composition of claim 31, wherein the melanopsin agonist comprises a compound having the structure: 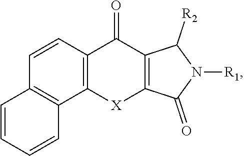
or a pharmaceutically-acceptable salt thereof.

34-40. (canceled)
